(19) United States
(12) Patent Application Publication
(10) Pub. No.: US 2008/0293623 A1
(43) Pub. Date: Nov. 27, 2008

(54) ENRICHED HAPTOGLOBIN POLYMERS FOR THE TREATMENT OF DISEASE

(75) Inventors: Joan Dalton, Berkhampsted Herts (GB); Adrian Podmore, Watford (GB)

Correspondence Address: KNOBBE MARTENS OLSON & BEAR LLP, 2040 MAIN STREET, FOURTEENTH FLOOR, IRVINE, CA, 92614, US

(73) Assignee: NHS Blood and Transplant, Watford (GB)

(21) Appl. No.: 12/113,036

(22) Filed: Apr. 30, 2008

Related U.S. Application Data
(60) Provisional application No. 60/924,090, filed on Apr. 30, 2007.

(51) Int. Cl. A61K 38/17; C07K 14/46; A61P 9/00; A61P 31/00

(52) U.S. Cl. 514/8; 530/392

(57) ABSTRACT

Haptoglobin (Hp) removes cell-free Hemoglobin (Hb), with different physiological effects depending on the particular Hp polymer. We propose that material enriched for alpha 1 chain Hp polymeric forms, such as those made from Cohn fraction V precipitate, will be more suitable for the treatment of certain diseases benefiting from both an antioxidant and anti-inflammatory component, such as for hemolytic disease. Material enriched for alpha-2 chain Hp polymeric forms, made for example from Cohn fraction IV precipitate, will be more suitable for treatment of diseases where an angiogenic, and/or inflammatory affect, and/or limited extravasation is desirable.

### RELATED APPLICATIONS

This application claims the benefit of U.S. Provisional Application No. 60/924,090 filed Apr. 30, 2007, which is hereby expressly incorporated by reference in its entirety.

### BACKGROUND OF THE INVENTION

1. Field of the Invention

The present application is concerned with the use of haptoglobin polymers or mixtures of polymers to treat medical conditions on the basis of the molecular and physiological activity of different polymeric forms of haptoglobin.

2. Description of the Related Art

Haptoglobin (Hp) is plasma protein. Hp is translated from a single mRNA, and the resultant peptide chain is cleaved to produce an alpha and a beta chain. Two main alleles are present in human populations, designated Hp1 and Hp2. The Hp2 gene is the product of non homologous crossing over of two Hp1 genes. This results in two different alpha chains designated alpha-1 and alpha-2, whereas there are no substantial differences in the beta chain. The beta chain (245 amino acids) has a mass of about 40 kDa (of which approximately 30% w/w is carbohydrate) and is shared by all phenotypes. The alpha-2 chain (142 amino acids) has a portion of the alpha-1 sequence repeated, and as such is approximately 16 kDa in weight whereas the alpha-1 chain (83 amino acids) is approximately 9 kDa in weight. Two co-dominant Hp alleles in Hardy Weinberg equilibrium result in three Hp phenotypes designated Hp1-1, Hp2-1 and Hp2-2. Hp from Hp1-1 individuals contains alpha-1 chains, that from Hp2-2 individuals alpha-2 chains, and that from Hp2-1 individuals contains both alpha-1 and one alpha-2 chains.

A single alpha and beta chain of Hp form a monomer unit. Each monomer unit can bind one alpha-beta chain unit of hemoglobin (Hb). The alpha-1 chain, the only alpha chain present in Hp1-1, can form one intermonomer bond. In Hp1-1 individuals, two monomer units form a dimer (approximately 90 kDa). Hp1-1 therefore exists as a single isoform, and is also referred to as Hp dimer. The alpha-2 chain, by virtue of its repeated alpha-1 sequence region, is able to form two such intermonomer bonds. Therefore, in Hp2-1 and Hp2-2 phenotypes larger Hp polymers are produced, with the largest polymers being present in Hp2-2 where two alpha 2 chain alleles are present. Hp2-1 has an average molecular mass of 220 kDa and forms liner polymers. Hp2-2 has an average molecular mass of 400 kDa and forms cyclic polymers. Each different polymeric form is a different isoform.

Discussion regarding the action of Hp in the literature has focused mainly on its ability to bind cell free hemoglobin (Hb). The conventional view of the action of Hp is as a kidney protector, binding to Hb to avoid its renal filtration and damaging effects to the kidney. The damaging effects of free HB are wide ranging and are shown diagrammatically in FIG. 1. Hp has been used as a treatment for conditions where there is a large amount of red cell lysis, such as third degree burns and transfusion reactions, in order to protect kidney function. The Green Cross Corp. of Japan produced Hp from plasma using fraction IV, IV-1 or IV-4 precipitate (U.S. Pat. No. 4,061,735) obtained according to Cohn's low temperature alcohol fractionation method as starting material (Cohn E. J., Strong L. E., Hughes W. L., Mulford D. J., Ashworth J. N., Melin M. and Taylor H. L (1946), Journal of the American Chemical Society, Vol. 68, pp 459-475). However, there is no evidence in the literature that the Green Cross Corp. considered the importance of the different molecular forms of Hp present in their preparations.

All Hp polymers have the same affinity for Hb. However, the Hb binding capacity is greater for smaller forms because of the greater molecular weight of the alpha-2 chain compared to the alpha-1 chain, meaning larger polymers containing more alpha-2 chain cannot bind as much Hb gram per gram. Some steric hindrance may also occur in larger polymers preventing total saturation of Hb binding sites.

WO03/006668 describes techniques for the generation of Hp derived peptides with antioxidant activity. However, peptides of Hp may not be as effective as preparations containing whole Hp molecules as they only provide one of the many beneficial activities of whole molecules of Hp. WO2006/107708 proposes use of anti-Hb antibodies to complex cell-free Hb. This will give some local protection from the toxic effects of cell-free Hb. However, the antibody-Hb complex will not be cleared by the Hp-Hb clearance mechanism and is unlikely to produce the same effects. Furthermore, the route taken to clear the antibody-Hb complex is unknown and may produce undesired effects. There may also be an adverse effect from the administration of anti-Hb antibodies.

Although Hp is generally known for its ability to remove cell-free Hb, it has many other biological effects either alone or once complexed to Hb. Some of the effects of Hp arise from the binding of Hp-Hb complex to macrophage receptor CD163. The activation of this receptor is known to result in stimulating the growth, proliferation, differentiation and/or mobilization of stem and/or progenitor cells (WO2006/094402, US-A 2004151692), and modification of the immune response (US-A 2005214871). It has also been proposed (US-A 2005063951) that the Hp-Hb complex could be used to target therapies to specific cell types that express CD163. A drug could be linked to the complex and administered to the patient. However, agents that target this receptor will again produce some but not all of the effects of Hp.

### SUMMARY OF THE INVENTION

We have analyzed and identified different combinations of Hp polymers in different fractions of plasma. In particular, we have found Hp in Cohn fraction V precipitate as well as Cohn fraction IV precipitate as used by the Green Cross Corp. These preparations, useful in that they can be manufactured from existing commercially generated plasma fractions as part of Cohn's low temperature alcohol fractionation method or modifications thereof, will have different biological properties. The present invention provides formulations of Hp enriched in one or more polymeric forms for use to treat or prevent certain diseases such as hemolytic anemia, sickle cell disease, cerebral vasospasm, organ failure, or other conditions where cell free Hb is present and changes in the vasculative, oxidative or inflammatory state are beneficial to a patient.

### BRIEF DESCRIPTION OF THE DRAWINGS

FIG. 1 illustrates the potential detrimental effects of hemolysis.

FIG. 2 illustrates the different potential physiological effects of dimer, trimer and larger forms of haptoglobin.

FIG. 3 illustrates a process for the production of an enriched haptoglobin formulation.

### DETAILED DESCRIPTION OF THE PREFERRED EMBODIMENT

In the future, phenotyping/genotyping patents for Hp may become more widespread in order to match Hp phenotype to the therapeutic intervention with the best chance of success (pharmacogenomics). We provide formulations of Hp enriched in one or more of the different polymeric forms which are targeted for use in the treatment of different conditions depending on the biological effects of the different Hp polymers. We are able to generate preparations from human plasma enriched for specific forms of Hp. Alternatively they can be generated using recombinant techniques.

As used herein, for plasma derived Hp polymeric formulations, “enriched” means that the formulation contains a higher % by weight (based on the total weight of Hp polymers) of at least one Hp polymer than was present in the starting human plasma. “Enriched” also includes formulations of substantially a single polymeric form, whether plasma derived or prepared by recombinant methods. Formulations containing mixtures of different polymeric forms derived from recombinant Hp are also covered by the term “enriched”.

As used herein, an “Hp polymer” contains more than one monomer unit formed from a single alpha chain and a single beta chain. The term polymer therefore includes dimers and trimers as well as higher polymeric forms.

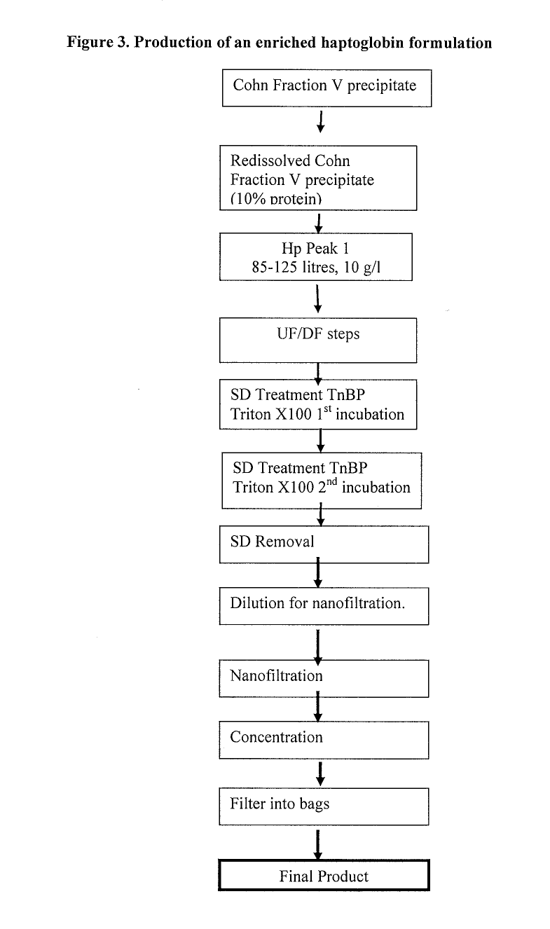

Hp may be prepared from Cohn fraction IV precipitate using affinity chromatography or anion exchange chromatography, or by the method of U.S. Pat. No. 4,061,735, the subject matter of which is hereby incorporated by reference. Hp may be prepared from Cohn fraction V precipitate using the method of WO2006/043062, the subject matter of which is hereby incorporated by reference. An Hp component purity of at least 65-70% can be achieved using this method, where the majority of other proteins present are zinc alpha-2 glycoprotein, alpha-1 beta glycoprotein and afamin. A preferred method starting from Cohn Fraction V precipitate is illustrated schematically in FIG. 3. In FIG. 3, UF refers to ultrafiltration, DF to diafiltration, SD to solvent detergent and TnBP to tri-n-butyl phosphate.

Any plasma derived Hp will preferably undergo at least one, more preferably two, and most preferably three, virus reduction and/or inactivation treatments prior to administration to a patient. Suitable virus reduction and/or inactivation treatments are known in the art and include dry heat treatment, pasteurization, virus filtration (also known as nanofiltration) and solvent-detergent treatment. A preferred combination of treatments is solvent-detergent treatment and virus filtration.

We have found that Hp derived from Cohn fraction IV precipitate tends to be lacking in the smaller Hp polymeric forms. By “smaller polymeric forms” is meant those of lower molecular weight containing primarily the alpha-1 chain, and including the dimeric and trimeric forms. Such forms typically have an average molecular weight below approximately 150 kDa. Thus, formulations derived from Cohn fraction IV precipitate are enriched for the larger Hp polymeric forms. By “larger polymeric forms” is meant those containing higher molecular weight polymers containing primarily the alpha-2 chain. Such polymeric forms typically have an average molecular weight above about 350 kDa. These larger polymeric forms of Hp can be isolated from the Cohn fraction IV precipitate fraction, for example by affinity chromatography or anion exchange chromatography.

Conversely, Hp derived from Cohn fraction V precipitate according to the method of WO2006/043062 tends to be richer in the smaller (low molecular weight) Hp polymeric forms. A preferred formulation isolated from Cohn fraction V precipitate according to the method of WO2006/043062 comprises approximately 46% dimer, 16% timer, 12% tetramer and 10% higher polymeric forms.

In a pharmaceutical formulation enriched in the smaller Hp polymeric forms, a majority (greater than 50%) of the alpha chains in the Hp are preferably of the alpha-1 type. In a non-limiting embodiment, formulations enriched in the smaller Hp polymeric forms will substantially comprise the dimeric and/or trimeric forms.

In a pharmaceutical formulation enriched in the larger Hp polymeric forms, a majority (greater than 50%) of the alpha chains in the Hp are preferably of the alpha-2 type.

Formulations enriched in intermediate Hp polymeric forms may also be prepared from Cohn fraction IV precipitate, Cohn fraction V precipitate, or mixtures thereof. By “intermediate polymeric forms” is meant those containing both the alpha-1 chain and the alpha-2 chain. Such polymeric forms typically have an average molecular weight of about 200 to about 300 kDa.

Recombinant Hp may be manufactured in suitable cell lines, preferably immortalized mammalian cell lines. Cell lines expressing a stable transcript of Hp1 cDNA can be used to produce recombinant dimeric Hp that should have similar properties to Hp derived from Cohn fraction V precipitate. Cell lines expressing a stable transcript of Hp2 cDNA may be used to produce recombinant large polymeric Hp (average molecular weight>350 kDa), that should have similar properties to Hp derived from Cohn fraction IV precipitate. Cell lines expressing a stable transcript of Hp2 and Hp1 cDNA may be used to produce the desired intermediate polymeric Hp polymeric forms. Mixtures containing different proportions of the different Hp polymeric forms could be made by mixing different recombinant forms in any desired proportion, or by controlling the mixture of cells used to produce the recombinant Hp.

Formulations of Hp enriched in smaller Hp polymeric forms may be useful in the treatment of conditions were vasoconstriction, inflammation and/or oxidation are having deleterious effects and/or presenting as clinical conditions. Thus, formulations of Hp enriched in the smaller Hp polymeric forms may be useful for the treatment of a disease or condition selected from the group: sickle cell disease, thalassemia, hereditary spherocytosis, hereditary stomatocytosis, microangiopathic hemolytic anemia, pyruvate kinase deficiency, paroxysmal cold hemoglobinuria, sever idiopathic autoimmune hemolytic anemia, infection-induced anemia, and malaria, and for the treatment of patients undergoing dialysis.

Formulations of Hp enriched in smaller Hp polymeric forms may also be useful to bring about resolution of cerebral vasospasm or for protection from it. Treatment could be either mediated by direct addition of the Hp formulation to the affected site via surgery, or via non-surgical intervention where a therapeutically effect amount of Hp would be given systemically. Systemic administration could be especially useful in preventing cerebral vasospasm after subarachnoid hemorrhage in individuals that carry an Hp2 allele.

Formulations of Hp enriched in larger forms of Hp may be useful in the treatment of conditions where an increased inflammatory response is required. Such conditions include but are not limited to systemic infection. It may be preferable to administer the larger forms of Hp as a complex with Hb.

Formulations of Hp enriched in larger forms of Hp and formulations of Hp enriched in intermediate Hp polymeric forms may be useful to stimulate angiogenesis, for example to stimulate production of coronary artery collaterals.

The invention also provides a method of treating a patient suffering from a condition or disease selected from the group consisting of: sickle cell disease, thalassemia, hereditary spherocytosis, hereditary stomatocytosis, microangiopathic hemolytic anemia, pyruvate kinase deficiency, paroxysmal cold hemoglobinuria, severe idiopathic autoimmune hemolytic anemia, infection-induced anemia, cerebral vasospasm and malaria, or treatment of a patient requiring dialysis, comprising the step of administering to said patient a therapeutically effective amount of a formulation of Hp enriched in smaller Hp polymeric forms.

The invention also provides a method of treating a patient suffering from systemic infection comprising the step of administering to said patient a therapeutically effective amount of a formulation of Hp enriched in larger Hp polymeric forms.

The invention also provides a method of stimulating angiogenesis in a patient comprising the step of administering to said patient a therapeutically effective amount of a formulation of Hp enriched in larger and/or intermediate Hp polymeric forms.

As used herein, “treatment” comprises both therapeutic and prophylactic (preventative) treatment. Therapeutic treatment may diminish, ameliorate or stabilize an existing disease or the side effects thereof.

By “formulation” is meant any pharmaceutically acceptable preparation of Hp suitable for administration to a human patient. Suitable formulations include, but are not limited to, aqueous solutions of Hp. Administration to a patient may be by any suitable route. Preferred routes of administration include, but are not limited to, intravenous administration and inhalation. For the treatment of certain conditions, direct application of the formulation to the affected site, for example via surgery, may be beneficial.

A preferred formulation for intravenous administration comprises Hp and an aqueous buffer. The pH of such a formulation is preferably in the range of about 5 to about 9, more preferably about pH 6.5 to about pH 7.5. Preferably the buffer contains less than about 12 mmol/L of phosphate ions to avoid the introduction of large amounts of phosphate when large doses of the formulation are administered to a patient.

Suitable dosage regimens will depend on the patient and condition being treated and on the mode of administration. In chronic conditions such as sickle cell anemia, repeated treatments will be necessary. For other conditions, a single treatment may be sufficient.

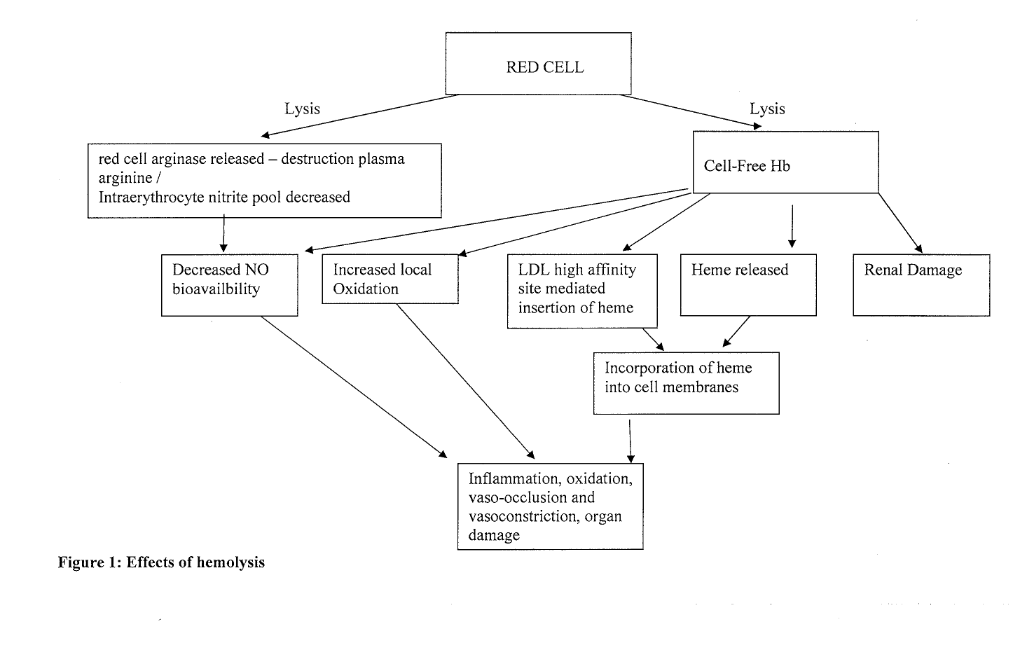

Hp is present in normal individuals at a concentration of 0.5-2 mg/mL (Langlois M R, Delanghe J R, Biological and clinical significance of haptoglobin polymorphism in humans, Clin Chem. 1996; 42: 1589-1600). However, even in cases of moderate hemolysis the amount of cell-free Hb released from lysed erythrocytes (red blood cells) can exceed the Hp binding capacity. In a normal otherwise healthy individual, quick removal of small amounts of Hb from the circulation is accounted for by Hp, and any Hb not accounted for by Hp is removed as heme by hemopexin (Hx). However, high cell-free Hb levels>1-3 g/L exceed this system's removal capacity and oxidative damage, as a result of systemic cell-free Hb, occurs. Protection against oxidative damage, as a result cell-free Hb exceeding Hp and Hx removal capacity, is effected by the activation of certain cellular mechanisms. This is done in large part by CD163 receptor clearance of Hb-Hp complexes. This acts as an activating signal as CD163 receptors are on monocytes (in circulation) and macrophages (in tissue). Thus, Hp therapy in instances where cell-free Hb has exceeded the capacity of the Hp/Hx system should be beneficial. Possible effects of failure to remove cell free Hb are shown in FIG. 1. In addition to binding intravascular Hb and thereby avoiding systemic oxidative damage, Hp also aids extravascular removal of Hb from the circulation by virtue of its ability to extravasate. Further the Hp-Hb complex formed signals the release of cytokines and alters levels of enzymes that can alter adverse events in inflammatory and oxidative states.

CD163 also binds and clears Hb, but the affinity of Hb for CD163 is 100 times lower than its affinity for Hp-Hb. Hp which is not bound to Hb does not bind CD 163. Further, it has been shown in vitro that Hp-Hb scavenger receptor internalizes the dimer (the only form of Hp found in Hp1-1 individuals) much more quickly than larger forms present in Hp2-2 individuals (Asleh R, et al. 2003 Genetically determined heterogeneity in hemoglobin scavenging and susceptibility to diabetic cardiovascular disease Circulation Research 92: 1193-1200).

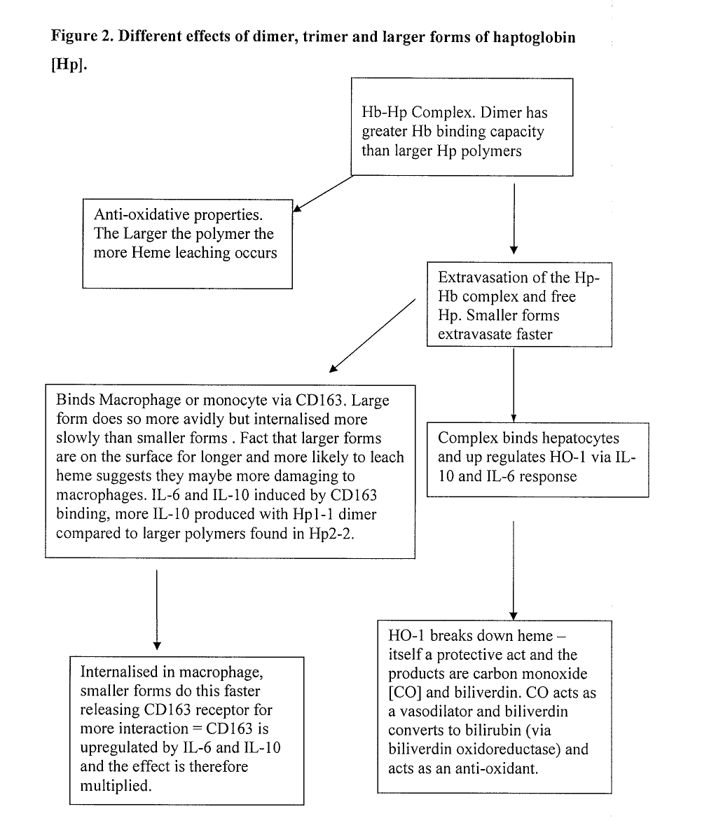

Hp itself is cleared from the circulation alongside the cell free Hb it has bound, and both molecules are degraded. A level of cell-free Hb that exceeds the Hp removal capacity does not stimulate Hp synthesis. This suggests that Hp is not intended as a means of removing all cell-free Hb in such instances of large hemolysis. Hemopexin is the other specific mechanism of dealing with cell-free Hb, but it is also insufficient to deal with a large amount of Hb because of its relatively low plasma concentration (0.5-0.7 mg/mL). Again the synthesis of hemopexin is not stimulated by levels of Hb or heme that exceed the Hp/hemopexin binding capacity, and although it is mostly recycled by the body, hemopexin Hb/heme removal capacity is quite limited. In such instances that both Hp and hemopexin capacity is exceeded albumin can bind free heme that results. However, albumin itself is a poor antioxidant and removal molecule compared to Hp and hemopexin, and hence oxidative damage occurs and Hb itself is recovered by the kidney that can cause renal iron loading and kidney damage. We contend therefore that the Hp-Hb complex has activities that alert cell targets to the hemolytic condition and provide responses via production of cytokines and enzymes. These activities, and the ability of different forms of Hp to affect them, are summarized in FIG. 2.

The smaller Hp polymeric forms are better at crossing the endothelial barrier than are the larger forms, leading to higher levels of the smaller forms in extravascular fluid (Mouray H, Moretti J, Jayle M F (1968), Haptoglobin exchange between intra- and extravascular spaces in rabbits, Bull Sco Chim Biol (article in French) (Paris) 50(4): 739-50; Tsuda-Kawamura K, Ogawa A, Tachibana N, Ohokubo H, Shibata K, Yanase T (1979), Type-dependent difference in the metabolism of human haptoglobins, Jap. J. Human Genet. 24: 85-94; Krauss S (1969), Haptoglobin metabolism in polycythemia vera, Blood vol 33 pp 865-876). Formulations enriched in smaller Hp polymeric forms may therefore exert beneficial therapeutic effects by promoting increased extravasation of Hp or the Hb-Hp complex. Increased extravasation of Hp will result in provision of Hp to remote non-vascular sites where the removal of cell free Hb is advantageous. Conditions in which removal of cell free Hb may be advantageous for a patient include, but are not limited to, sickle cell disease, thalassemia, hereditary spherocytosis, hereditary stomatocytosis, pyruvate kinase deficiency, paroxysmal cold hemoglobinuria, severe idiopathic autoimmune hemolytic anemia, and cerebral vasospasm. It may also be beneficial for patients requiring dialysis. Where it is beneficial to retain Hp in the circulation, larger forms may be more suitable. Conditions in which it may be beneficial to retain Hp in the circulation include, but are not limited to, microangiopathic hemolytic anemia, ABO mismatch transfusion reaction, infection-induced anemia, malaria, and mechanical heart valve-induced anemia. It may also be beneficial for patients undergoing cardiopulmonary bypass.

Formulations of Hp enriched in the smaller Hp polymeric forms, especially the dimeric form, tend to have better antioxidative ability than the larger forms (Grinshtein N, Bamm V V, Tsemakhovich V A, Shaklai N (2003), Mechanisms of low-density lipoprotein oxidation by hemoglobin derived iron, Biochemistry. 42(23): 6977-6985; Bamm V V, Tsemakhovich V A, Shaklai M, Shaklai N (2004), Haptoglobin phenotypes differ in their ability to inhibit heme transfer from hemoglobin to LDL, Biochemistry 43(13):3899-3906; Melamed-Frank M, Lache O, Enav B I, Szafranek T, Levy N S, Ricklis R M, Levy A P (2001), Structure-function analysis of the antioxidant properties of haptoglobin, Blood 98: 3693-3698; Asleh R, Guetta J, Kalet-Litman S et al (2005), Haptoglobin genotype- and diabetes-dependent differences in iron-mediated oxidative stress in vitro and in vivo, Circ Res vol 96 pp 435-441; Gueye P M, Glasser N, Ferard G, Lessinger J M (2006), Influence of human haptoglobin polymorphism on oxidative stress induced by free hemoglobin on red blood cells, Clin Chem Lab Med vol 44 no 5 pp 542-547). Smaller Hp polymeric forms also produce greater anti-inflammatory effects. Binding of haptoglobin-hemoglobin complexes to CD163 can upregulate the expression of this CD receptor, release the pro-inflammatory cytokine IL-6, and on internalization of the complex induce expression of the anti-inflammatory cytokine IL-10. Clearance of Hp-Hb complexes results in cytokine mediated upregulation of CD163 via IL-6 and IL-10 (Philippidis P, Mason J C, Evans B J, Nadra I, Taylor K M, Haskard D O, Landis R C (2004), Hemoglobin scavenger receptor CD163 mediates interleukin-10 release and heme oxygenase-1 synthesis: anti-inflammatory monocyte-macrophage responses in vitro, in resolving skin blisters in vivo, and after cardiopulmonary bypass surgery, Circ Res 94: 119-126; Maniecki M B, Moller H J, Moestrup S K, Moller B K., CD163 positive subsets of blood dendritic cells: the scavenging macrophage receptors CD163 and CD91 are coexpressed on human dendritic cells and monocytes, Immunobiology. 2006; 211:407-417; Levy A P. Application of pharmacogenomics in the prevention of diabetic cardiovascular disease: Mechanistic basis and clinical evidence for utilization of the haptoglobin genotype in determining benefit from antioxidant therapy, Pharmacol Ther. 2006; 112:501-512). It has been shown in vitro that Hp-Hb scavenger receptor internalizes the dimer present in Hp1-1 much more quickly than the larger forms present in Hp2-2 (Asleh R, Marsh S, Shillrut M, Binah O, Guetta J, Lejbkowicz F, Enav B, Shehadeh N, Kanter Y, Lache O, Cohen O, Levy N S, Levy A P (2003), Genetically determined heterogeneity in hemoglobin scavenging and susceptibility to diabetic cardiovascular disease, Circulation Research 92: 1193-1200) leading to the dimer producing a greater anti-inflammatory effect than the larger forms of haptoglobin.

It was also recently found that an Hp2 allele predisposes to a cerebral vasospasm after a subarachnoid hemorrhage (Borsody M, Burke A, Coplin W, Miller-Lotan R, Levy (2006), Haptoglobin and the development of cerebral artery vasospasm after subarachnoid hemorrhage, Neurology 66(5):634-640). This is likely to be due to the lower extravasation potential and lower antioxidative activity of larger polymeric forms of Hp. Administration of formulations enriched in smaller Hp polymeric forms, and in particular the dimer, are therefore likely to be particularly beneficial for the treatment or prevention of cerebral vasospasm in individuals possessing an Hp2 allele.

Haptoglobin clears hemoglobin via CD163 receptors. This causes the production of IL-10 and IL-6 that both stimulate the production of the enzyme HO 1. The HO-1 has both systemic and tissue-protective effects as it leads to the production of CO and biliverdin from heme. Biliverdin reductase converts biliverdin to bilirubin. Bilirubin and carbon monoxide are both powerful anti-oxidative and anti-inflammatory agents. Carbon monoxide is also a vasodilator. Therefore enhanced HO-1 production would be beneficial in such indications as: sickle cell disease, thalassemia, hereditary spherocytosis, hereditary stomatocytosis, microangiopathic hemolytic anemia, pyruvate kinase deficiency, ABO mismatch transfusion reaction, paroxysmal cold hemoglobinuria, severe idiopathic autoimmune hemolytic anemia, infection-induced anemia, malaria, cerebral vasospasm, and mechanical heart valve-induced anemia, or in patients requiring dialysis or cardiopulmonary bypass.

As a result of the properties of Hp referred to above, we propose use of formulations of Hp enriched in the smaller Hp polymeric forms in the treatment of conditions where increased extravasation and/or anti-inflammatory and/or anti-oxidant effects are expected to be beneficial, including but not limited to sickle cell disease, thalassemia, hereditary spherocytosis, hereditary stomatocytosis, microangiopathic hemolytic anemia, pyruvate kinase deficiency, paroxysmal cold hemoglobinuria, severe idiopathic autoimmune hemolytic anemia, infection-induced anemia, malaria, and cerebral vasospasm, and in patients requiring dialysis.

The larger Hp polymers have enhanced inflammatory properties and reduced extravasation properties when compared to the smaller Hp polymers. Thus, we propose the use of formulations enhanced in the larger Hp polymers in the treatment of conditions such as infections where an enhanced inflammatory response would be beneficial to the patient. In addition, we propose the use of larger polymers of Hp (>400 kDa) to stimulate angiogenesis such as the production of coronary artery collaterals. It has been shown (Lohr N L, Warltier D C, Chilian W M, Weihrauch D (2005), Haptoglobin expression and activity during coronary collateralization, Am J Physio Heart Circ Physiol vol 288 pp H1389-H1395) that Hp plays a critical role during coronary artery collateral formation, and formation was stimulated in repetitive occlusive events. Therefore, where patients can withstand any inflammatory side effects, doses of large polymeric Hp (>400 kDa) may well be beneficial to the development of coronary artery collaterals.

In certain diseases with an Hb oxidative component such as diabetes others have reported more beneficial coronary artery collaterals with Hp2-1 compared to Hp2-2 (Hochberg I, Roguin A, Nikolsky E et al (2002), Haptoglobin phenotype and coronary artery collaterals in diabetic patients, Atherosclerosis vol 161 pp 441-446). In this instance we propose that use of an Hp preparation of a mixture of alpha-1 chain and alpha-2 chain containing components may well give the best performance due to the beneficial anti-inflammatory and antioxidative protection afforded by alpha-1 chain containing Hp, combined with the angiogenic ability of the alpha-2 chain containing Hp.

While the above description contains many specific details of methods and formulations in accordance with the invention, these specific details should not be construed as limitations on the scope of the invention, but merely as exemplifications of preferred embodiments thereof. Those skilled in the art will be able to envisage many other possible variations that fall within the scope and spirit of the invention as defined by the claims appended hereto.

It is understood that the examples and embodiments described herein are for illustrative purposes only and that various modifications or changes in light thereof will be suggested to persons skilled in the art and are to be included within the spirit and purview of this application and scope of any appended claims. All figures, tables, and appendices, as well as publications, patents, and patent applications, cited herein are hereby incorporated by reference in their entirety for all purposes.

What is claimed is:

1. A pharmaceutical formulation comprising haptoglobin [Hp] enriched in at least one polymeric form.

2. The pharmaceutical formulation according to claim 1, comprising Hp enriched in smaller polymeric forms.

3. The pharmaceutical formulation according to claim 2, which in liquid form has a pH between about 6.5 and about 7.5 and comprises a buffer that contains no more than about 12 mmol/L phosphate ions.

4. The pharmaceutical formulation according to claim 2 wherein the Hp is derived from Cohn fraction V precipitate.

5. The pharmaceutical formulation according to claim 2 wherein the Hp is recombinant.

6. The pharmaceutical formulation according to claim 2 wherein a majority of alpha chains in the Hp are of the alpha-1 type.

7. A method of treating a patient suffering from a disease selected from the group consisting of: sickle cell disease, thalassemia, hereditary spherocytosis, hereditary stomatocytosis, microangiopathic hemolytic anemia, pyruvate kinase deficiency, paroxysmal cold hemoglobinuria, severe idiopathic autoimmune hemolytic anemia, infection-induced anemia, malaria, and cerebral vasospasm, comprising the step of administering to said patient a therapeutically effective amount of the formulation of claim 2.

8. A method of treating a patient or patients requiring dialysis, comprising the step of administering to said patient a therapeutically effective amount of the formulation of claim 2.

9. A method of promoting extravasation of Hp or Hb-Hp complexes in a patient comprising the step of administering to said patient a therapeutically effective amount of the formulation of claim 2.

10. A method of promoting production of IL-10 in a patient comprising the step of administering to said patient a therapeutically effective amount of the formulation of claim 2.

11. A method of promoting production of HO-1 enzyme in a patient comprising the step of administering to said patient a therapeutically effective amount of the formulation of claim 2.

12. A method of reducing the amount of heme leaching in a patient comprising the step of administering to said patient a therapeutically effective amount of the formulation of claim 2.

13. A method of protecting a patient against cerebral vasospasm comprising the step of administering to said patient a therapeutically effective amount of formulation of claim 2.

14. The method according to claim 12 wherein the patient carries an Hp2 allele.

15. The pharmaceutical formulation according to claim 1, comprising Hp enriched in larger polymeric forms.

16. The pharmaceutical formulation according to claim 14 wherein the Hp has an average molecular weight of greater than about 350 kDa.

17. The pharmaceutical formulation according to claim 14 wherein a majority of alpha chains in the Hp are of the alpha-2 type.

18. The pharmaceutical formulation according to claim 14 wherein the Hp is derived from Cohn fraction IV precipitate.

19. The pharmaceutical formulation according to claim 14 wherein the Hp is recombinant.

20. A method of protecting organ function in a patient during acute hemolysis occurring as a result of surgery, or hemolysis occurring as a result of some other insult, comprising the step of administering to said patient a therapeutically effective amount of the formulation of claim 15.

21. A method of promoting improvement of vasculature, or to improve development of coronary artery collaterals or other such vessel formation in a patient, comprising the step of administering to said patient a therapeutically effective amount of the formulation of claim 15.

22. A method of promoting healing of chronic wounds in a patient such as chronic venous ulcers, acute wounds or mechanical heart valve-induced anemia comprising the step of administering to said patient a therapeutically effective amount of the formulation of claim 15.

23. A method of treating a patient suffering from systemic infection comprising the step of administering to said patient a therapeutically effective amount of the formulation of claim 15.

24. A method of stimulating angiogenesis in a patient comprising the step of administering to said patient a therapeutically effective amount of the formulation of claim 15.
